(19) United States
(12) Patent Application Publication
(10) Pub. No.: US 2009/0047239 A1
(43) Pub. Date: Feb. 19, 2009

(54) Stabilised IL-21 Compositions

(75) Inventors: Malin Persson, Malmö (SE); Jens Thostrup Bukrinski, Valby (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: NOVO NORDISK A/S, BAGSVAERD, DK (unknown)

(21) Appl. No.: 11/916,674

(22) PCT Filed: Jun. 6, 2006
(86) PCT No.: PCT/EP2006/062920

§ 371 Date: Mar. 4, 2008

Related U.S. Application Data
(60) Provisional application No. 60/689,005, filed on Jun. 9, 2005.

(30) Foreign Application Priority Data
Jun. 6, 2005 (EP) 05104887.4

(51) Int. Cl. A61K 45/00; A61P 31/00

(52) U.S. Cl. 424/85.2

(57) ABSTRACT

Compositions comprising IL-21 and sulphate are provided.

### FIELD OF THE INVENTION

The present invention relates to compositions, e.g. pharmaceutical compositions comprising IL-21 and sulphate, and to methods of stabilising IL-21, the method comprising adding sulphate to a solution comprising said peptide.

### BACKGROUND OF THE INVENTION

IL-21 was first disclosed in WO 00/53761. The pro-peptide is a 162 amino acid residue peptide, the sequence of which is disclosed as SEQ ID No: 2 in said application. For convenience, the sequence is repeated in this application as SEQ ID No: 1. It was initially believed that the mature peptide was the peptide consisting of amino acid no 32 to 162 of SEQ ID No: 1, however more recently (WO 04/112703) is has been suggested that the mature peptide is, in fact, amino acid no 30 to 162.

IL-21 is a cytokine capable of activating B-cells, T-cells, and NK cells, and IL-21 and variants and analogues thereof have been suggested as therapeutics effective to treat various cancer forms, WO 00/53761 and WO 03/103589.

Peptides are normally not stable in liquid form over extended periods of time, and a lot of research is invested in providing formulations of pharmaceutical peptides which fulfils the stability requirement to pharmaceutical compositions. The provision of stable pharmaceutical compositions comprising peptides is not trivial, and it thus remains a challenge to find ways to provide stable formulations comprising. IL-21. The present invention aims at providing such stable formulations or compositions with alternative or improved properties.

WO 04/112703 discloses that e.g. amino acid 30 to 162 of SEQ ID No: 1 may be isolated by methods comprising precipitating the peptide with high concentrations of ammonium sulphate.

WO 04/055168 discloses that cells transfected with a vector comprising a poly nucleotide encoding amino acid no 30 to 162 of SEQ ID No: 1 with an additional N-terminal Met may be fermented in a medium comprising ammonium sulphate.

International application WO2004DK000686 (published as WO 05/35565 discloses the use of copper sulphate as catalyst in a reaction between a functionalised PEG and a functionalised IL-21 to provide a PEGylated IL-21.

### SUMMARY OF THE INVENTION

The present inventors have surprisingly found that the presence of sulphate ions

(SO₄²⁻) stabilises the structure of IL-21 and increases the stability of compositions comprising IL-21. Accordingly, in one embodiment, the present invention provides a composition, and in particular a pharmaceutical composition comprising IL-21 and sulphate ions, provided any ammonium ions (NH₄⁺) is not present in said composition in a molar amount which is twice the molar amount of sulphate.

In one embodiment, the invention provides a composition, and in particular a pharmaceutical composition comprising IL-21 and sulphate ions, provided any ammonium ions (NH₄⁺) is not present in said composition in a molar amount which is twice the molar amount of sulphate, and provided that the composition does not comprise copper.

In one embodiment, the invention provides a method of stabilising a composition comprising IL-21, the method comprising adding sulphate to said composition.

In one embodiment, the invention provides a method of folding or re-folding IL-21, the method comprising adding sulphate to unfolded or partially folded IL-21.

In one embodiment, the invention provides a method of purifying IL-21, the method comprising contacting chromatographic material with a composition comprising IL-21 and sulphate.

In one embodiment, the invention provides a method of crystallising IL-21, the method comprising adding sulphate to a composition comprising IL-21.

In one embodiment, the present invention relates to a therapeutic method, the method comprising the administration of an effective amount of a pharmaceutical composition of the present invention to a patient in need thereof.

In one embodiment, the present invention relates the use of IL-21 and sulphate in the preparation of a medicament.

In one embodiment, the present invention provides a pharmaceutical composition comprising IL-21 and sulphate ions.

### LEGENDS TO FIGURES

FIG. 1: 1-Anilinonaphtalene-8-sulfonic acid (ANS) binding to IL-21 at different pH. All samples contained 0.05 mg/ml (3.2 μM) IL-21, 10 mM buffer (which buffer and pH is shown in the figure) and when indicated, 50 mM Na₂SO₄. 16 μM ANS was added just before measurement.

FIG. 2: Far-UV CD spectra of IL-21 at pH 2.0 in phosphate buffer with and without 50 mM sulphate added. Protein concentration 10 mg/ml.

FIG. 3A: Far-UV CD spectra of IL-21 at pH 5.3 in histidine buffer with and without 50 mM sulphate added. Protein concentration 10 mg/ml.

FIG. 3B: Far-UV CD spectra of IL-21 at pH 5.3 in acetate buffer with and without 50 mM sulphate added. Protein concentration 10 mg/ml.

FIG. 4: Far-UV CD spectra of IL-21 at pH 6.0 in phosphate buffer with and without 50 mM sulphate added. Protein concentration 10 mg/ml.

FIG. 5: Far-UV CD spectra of IL-21 at pH 2.0 to 6.0 without sulphate added. Protein concentration is 10 mg/ml.

FIG. 6: Far-UV CD spectra of IL-21 at pH 2.0 to 6.0 with 50 mM sulphate added. Protein concentration is 10 mg/ml.

FIG. 7: Near-UV CD spectra of IL-21 at pH 2.0 in phosphate buffer with and without 50 mM sulphate added. Protein concentration 10 mg/ml.

FIG. 8A: Near-UV CD spectra of IL-21 at pH 5.3 in histidine buffer with and without 50 mM sulphate added. Protein concentration 10 mg/ml.

FIG. 8B: Near-UV CD spectra of IL-21 at pH 5.3 in acetate buffer with and without 50 mM sulphate added. Protein concentration 10 mg/ml.

FIG. 9: Near-UV CD spectra of IL-21 at pH 6.0 in phosphate buffer with and without 50 mM sulphate added. Protein concentration 10 mg/ml.

FIG. 10: Capillary DSC of IL-21 at pH 5.3 in histidine buffer with and without 50 mM Na-sulphate.

FIG. 11: The data of table 1 depicted in a plot.

FIG. 12: Near-UV CD spectra of IL-21 at pH 2 in phosphate, sulphate, acetate and formate buffers at 50 mM. Protein concentration 2 mg/ml.

### DEFINITIONS

A “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on e.g. the severity of the disease or injury as well as the weight, sex, age and general state of the subject. It will be understood that determining an appropriate dosage may be achieved using routine experimentation, by constructing a matrix of values and testing different points in the matrix, which is all within the ordinary skills of a trained physician or veterinary.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being, but it may also include animals, such as dogs, cats, cows, sheep and pigs. Nonetheless, it should be recognized that therapeutic regimens and prophylactic (preventative) regimens represent separate aspects of the invention.

### DESCRIPTION OF THE INVENTION

In this context, IL-21 is intended to indicate

a) a peptide with a sequence as defined in SEQ ID No: 1;
b) a peptide with a sequence defined by amino acid no 32-162 of SEQ ID No: 1;
c) a peptide with a sequence defined by amino acid no 30-162 of SEQ ID No: 1; or
d) a variant of any of a), b) and c).

A variant is understood to be the compound obtained by substituting one or more amino acid residues in the IL-21 sequence with another natural or unnatural amino acid; and/or by adding one or more natural or unnatural amino acids to the IL-21 sequence; and/or by deleting one or more amino acid residue from the IL-21 sequence, wherein any of these steps may optionally be followed by further derivatization of one or more amino acid residue. In particular, such substitutions are conservative in the sense that one amino acid residue is substituted by another amino acid residue from the same group, i.e. by another amino acid residue with similar properties. Amino acids may conveniently be divided in the following groups based on their properties: Basic amino acids (such as arginine and lysine), acidic amino acids (such as glutamic acid and aspartic acid), polar amino acids (such as glutamine, cysteine, histidine and asparagine), hydrophobic amino acids (such as leucine, isoleucine, proline, methionine and valine), aromatic amino acids (such as phenylalanine, tryptophan, tyrosine) and small amino acids (such as glycine, alanine, serine and threonine.).

In one embodiment, said variant has at least 80%, such as at least 85%, such as at least 90%, such as at least 95% identity with a), b) or c). In particular, said variants have at least 20%, such as at least 40%, such as at least 60%, such as at least 80%, such as at least 90%, such as at least 95% of the activity of a), b), or c) as determined in assay I herein.

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related proteins can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48:1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res., 12:387 (1984); Genetics Computer Group, University of Wisconsin, Madison, Wis.), BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two proteins for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp.3 (1978) for the PAM 250 comparison matrix; Henikoff et al., Proc. Natl. Acad. Sci. USA, 89:10915-10919 (1992) for the BLOSUM 62 comparison matrix) is also used by the algorithm.

Preferred parameters for a protein sequence comparison include the following:

Algorithm: Needleman et al., J. Mol. Biol, 48:443-453 (1970); Comparison matrix: BLOSUM 62 from Henikoff et al., Proc. Natl. Acad. Sci. USA, 89:10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

In one embodiment, the variant is obtained by adding up to five amino acids, such as one, two, three, four or five, to any of the sequences a), b) and c) above, and in particular to the N-terminal. Particular mentioning is made of c) (i.e. amino acid 30-162 of SEQ ID No: 1) with an additional Met added to the N-terminal, SEQ ID No: 2.

The invention also encompass compositions of the present invention comprising the above discussed IL-21 variants which have been further derivatised, e.g. by the covalent attachment of PEG or a lipophilic substituent, for instance as described in WO 05/035565.

In one embodiment, the compositions of the present inventions do not comprise ammonium in a molar amount which is twice the molar amount of sulphates. As ammonium is an acid, the amount of it will depend on the pH of the solution; at high pH the ammonium will be present as ammonia (NH₃). It is to be understood that in the present context the amount of ammonium is to be calculated as the total amount of ammonia and ammonium. In a similar manner, the amount of sulphate is dependent of the pH. At low pH, sulphate is present in the protonated form, HSO₄⁻. It is to be understood that in the present context, the amount of sulphate is to be calculated as the total amount of sulphate and the protonated form.

In one embodiment of the invention, the compositions of the present invention do not comprise ammonium. It is understood that this is intended to mean that the compositions of the present invention do not comprise copper above the trace amount level.

The sulphate ion used in the present invention may in principle come from any sulphate salt. Certain areas or industries may, of course, have special requirements to said salts which also have to be fulfilled. As an example, the pharmaceutical industry may require that the sulphate salt is a pharmaceutically acceptable salt. Pharmaceutically acceptable sulphate salts include metal salts, such as lithium-, sodium-, zinc-, calcium-, potassium-, and magnesium sulphate, and alkylted ammonium salts, such as methylammonium-, dimethylammonium-, trimethylammonium-, ethylammonium-, triethylammonium-, hydroxyethylammonium-, diethylammonium-, butylammonium-, and tetramethylammonium-sulphate. Some of the above mentioned salts exist in more than one form comprising different amounts of crystal-water. Any of these forms may be used in the present invention, and particular mentioning is made of sodium sulphate.

In one embodiment, a composition of the present invention does not comprise copper. It is understood that this is intended to mean that the compositions of the present invention do not comprise copper above the trace amount level.

In one embodiment, the present invention provides a pharmaceutical composition comprising IL-21 and sulphate.

In one embodiment, the present invention provides a pharmaceutical composition comprising IL-21 and sulphate, with the provision that ammonia is not present in a molar amount which is twice the molar amount of sulphate.

In one embodiment, the present invention provides a pharmaceutical composition comprising IL-21 and sulphate, with the provision that ammonia is not present in a molar amount which is twice the molar amount of sulphate, and with the further provision that the composition does not comprise copper. Besides IL-21 and sulphate, said pharmaceutical composition may also comprise other excipients known in the art, such as buffers, preservatives, isotonic agents, chelating agents, stabilizers, an amino acid base sufficient to decrease aggregate formation by the protein during storage of the composition, surfactants, wetting agents, emulsifiers, antioxidants, bulking agents, metal ions, oleaginous vehicles, proteins (e.g., human serum albumin, gelatine or proteins) and a zwitterions. The use of these excipients is well known to the person skilled in the art and described in e.g. Remington: The Science and Practice of Pharmacy, 20^{th} edition, 2000.

In one embodiment, the pharmaceutical compositions of the present invention do not comprise ammonium. It is understood that this is intended to mean that the compositions of the present invention do not comprise copper above the trace amount level.

A pharmaceutical composition of the present invention may be administered in several dosage forms, such as e.g. solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, e.g. lyophilised powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

The pharmaceutical composition of the present invention may be administered through several routes of administration, such as e.g. lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment. Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition of the present invention which may be a solution or suspension for administration in the form of a nasal or pulmonal spray. As a still further option, the pharmaceutical compositions of the present invention can also be adapted to transdermal administration, e.g. by needle-free injection or from a patch, optionally an iontophoretic patch, or transmucosal, e.g. buccal, administration.

In the pharmaceutical compositions of the present invention comprising IL-21 and sulphate, IL-21 may be present in amounts up to 500 mg/ml. In particular, IL-21 may be present at concentrations of or up to 5, 10, 20, 30, 40, 50, 60, 70, 80, 90 or 100 mg/ml. IL-21 is stabilised at sulphate concentrations above 1 mM, such as above 5 mM, such as above 7 mM, such as above 10 mM, such as above 20 mM. In fact, up to 500 mM, such as up to 150 mM, such as up to 75 mM, such as up to 50 mM sulphate may be present. The upper limit of the sulphate concentration is dictated by the solubility of the sulphate salt chosen. In many applications of the present invention, it is desirable to have a pharmaceutical composition which is isotonic, or near isotonic. In one embodiment, the pharmaceutical composition is from 20% isotonic to 120% isotonic.

In one embodiment, the sulphate concentration is from about 1 to about 100 mM. In one embodiment, the sulphate concentration is from about 20 to about 100 mM. In further embodiments, the sulphate concentration is selected from the group consisting of about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, and about 100 mM.

A pharmaceutical composition of the invention may for instance also be non-isotonic and may be intended to be made isotonic, for instance by dilution, prior to administration. This may enable the sale of more concentrated pharmaceutical compositions, which are then made isotonic by for instance the physician or the patient prior to administration.

In one embodiment of the invention chloride is added to the compositions of the present invention. In particular, up to 150 mM chloride may be added.

The pH of the pharmaceutical compositions of the present invention is preferably between 2 and 8, and particular mentioning is made of pharmaceutical compositions with pH between 4 and 6, such as e.g. 5.3.

An example of an invention of the present invention consist of 10 mg/ml of the peptide defined by SEQ ID No: 2, 10 mM Na₂SO₄, buffered with histidine at pH 5.3.

The impact of a particular excipient on the stability of pharmaceutical compositions is typically assessed by preparing a test composition (in casu a composition of the present invention) and a reference composition (in casu the same composition but without sulphate). The two compositions are then stored under controlled conditions, e.g. 2 weeks at 40° C., 2 months at 25° C. or 6 months at 5° C. After storage, the residual activity of the peptide of interest in the two compositions are measured in a suitable assay. For composition comprising IL-21, the aggregation assay described in example 11 herein may be used. Other assays measuring the amount/activity of IL-21 or degradation products may also be applied.

One embodiment of the present invention relates to the use of sulphate to promote folding or re-folding of IL-21 from un-folded or partially un-folded IL-21. In particular, addition of sulphate ions may increase the speed of folding or secure correct folding. If IL-21 is produced by fermentation of particular micro-organisms which are not able to fold the peptides correctly, the peptides may be expressed in exclusion bodies comprising un-folded or partially un-folded peptide. After disruption of said inclusion bodies, the correctly folded peptide may be obtained in processes including the addition of sulphate ions.

The presence of sulphate ions effects a more compact and stable structure of IL-21. This may be exploited in chromatographic purification of IL-21 to change the chromatographic elution profile of the peptide, and in particular to give a sharper and more well-defined elution peak. The beneficial effects of adding sulphate may been seen in different kinds of chromatography, such as ion exchange chromatography (cation exchange chromatography and anion exchange chromatography) and hydrophobic interaction chromatography, size exclusion chromatography. A sharper elution peak is advantageously in situations where separation for a close-eluting peak is desired, but also in general as the eluting peptide is then collected in a smaller volume which enables simpler and cheaper further down-stream processing. According to the invention, sulphate is present in at least around 3 mM, such as at least around 7, such as at least around 10 mM. In fact, it is anticipated that only the solubility of the applied sulphate salt defines an upper limit to the sulphate concentration. Particular mentioning is made of sodium sulphate as the source of sulphate.

The pH of the chromatographic processes of the present invention may be from 1 to 10, such as from 2 to 9, such as from 3 to 8, such as from 4 to 7, such as from 5 to 6.

Many different chromatographic materials are know in the art, and examples include anion exchange chromatography material, cation exchange chromatography material, hydrophobic interaction material and size exclusion chromatography material.

A more well-defined structure is more easy to crystallise, and the present invention therefore also provides the use of sulphate to improve crystallisation of IL-21. In this context, “improve” may be the increase of the crystallisation speed, or the provision of crystals which are more suitable for e.g. X-ray diffraction studies.

IL-21 has been suggested in the treatment of various cancers or neoplastic diseases or disorders. In one embodiment, the present invention relates to a method of treating cancer, the method comprising the administration of a therapeutically effective amount of a composition of the present invention comprising IL-21 and sulphate to a patient in need thereof. Particular mentioning is made of metastatic malignant melanoma, renal cell carcinoma, ovarian cancer, small-cell lung cancer, non small-cell lung cancer, breast cancer, colorectal cancer, prostate cancer, pancreatic cancer, bladder cancer, esophageal cancer, cervical cancer, endometrial cancer, lymphoma, and leukaemia.

In more specific aspects of the invention, the terms “neoplastic disorders”, “cancer” are to be understood as referring to all forms of neoplastic cell growth, including both cystic and solid tumors, bone and soft tissue tumors, including both benign and malignant tumors, including tumors in anal tissue, bile duct, bladder, blood cells, bone, bone (secondary), bowel (colon & rectum), brain, brain (secondary), breast, breast (secondary), carcinoid, cervix, children's cancers, eye, gullet (esophagus), head & neck, kaposi's sarcoma, kidney, larynx, leukaemia (acute lymphoblastic), leukaemia (acute myeloid), leukaemia (chronic lymphocytic), leukaemia (chronic myeloid), leukaemia (other), liver, liver (secondary), lung, lung (secondary), lymph nodes (secondary), lymphoma (hodgkin's), lymphoma (non-hodgkin's), melanoma, mesothelioma, myeloma, ovary, pancreas, penis, prostate, skin, soft tissue sarcomas, stomach, testes, thyroid, unknown primary tumor, vagina, vulva, womb (uterus).

Soft tissue tumors include Benign schwannoma Monosomy, Desmoid tumor, Lipo-blastoma, Lipoma, Uterine leiomyoma, Clear cell sarcoma, Dermatofibrosarcoma, Ewing sarcoma, Extraskeletal myxoid chondrosarcoma, Liposarcoma myxoid, Liposarcoma, well differentiated, Alveolar rhabdomyosarcoma, and Synovial sarcoma.

Specific bone tumor include Nonossifying Fibroma, Unicameral bone cyst, Enchon droma, Aneurysmal bone cyst, Osteoblastoma, Chondroblastoma, Chondromyxofibroma, Ossifying fibroma and Adamantinoma, Giant cell tumor, Fibrous dysplasia, Ewing's Sarcoma, Eosinophilic Granuloma, Osteosarcoma, Chondroma, Chondrosarcoma, Malignant Fibrous Histiocytoma, and Metastatic Carcinoma.

Leukaemias refers to cancers of the white blood cells which are produced by the bone marrow. This includes but are not limited to the four main types of leukaemia; acute lymphoblastic (ALL), acute myeloblastic (AML), chronic lymphocytic (CLL) and chronic myeloid (CML).

Moreover IL-21 can be used therapeutically in cancers of various non-metastatic as wells as metastatic stages such as “Stage 1” Localized (confined to the organ of origin); “Stage 2” Regional; “Stage 3” Extensive; and “Stage 4” Widely disseminated cancers.

IL-21 has also been implicated in the treatment of viral infections, such as hepatitis B Virus, Hepatitis C virus, Human Immunodeficiency Virus, Respiratory Syncytial Virus, Eppstein-Barr Virus, Influenza Virus, Cytomegalovirus, Herpes-Virus and Severe Acute Respiratory Syndrome.

In one embodiment, the present invention relates to the use of IL-21 and sulphate in the manufacture of a medicament for the treatment of cancer. Particular mentioning is made of cancers from the above list.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

### Assay 1

IL-21 activity may be analyzed in a cellular bioassay employing a murine Baf3 cells, stably transfected to express the human IL-21R and a Stat-linked luciferase reporter construct. The Baf3 cells expresses endogenously the gc component of the active IL-21 receptor complex. The Baf3/hIL-21R reporter cell line was starved in IL-3 free medium for 18 hours prior to stimulation. A dosis-response curve may be obtained using a range of concentrations of the IL-21 protein to calculate an IC₅₀ concentration. The Baf3 cells are described in Palacios R, Nature. 309(5964), 126-31 (1984) and are available from the Cell Bank at RIKEN BioResource Center (http://www2.brc.riken.jp/lab/cell/detail.cgi?cell_no=RCB0805).

### EXAMPLES

In the following examples, “IL-21” is meant to be the peptide identified by SEQ ID No:2.

### Example 1

### ANS Fluorescence

ANS (1-anilinonaphtalene-8-sulfonic acid) binds to hydrophobic sites in proteins. A stronger fluorescence intensity indicates that more ANS molecules are bound to the protein. This again is indicative of a less structured molecule because a less structured protein will expose more hydrophobic sites. A weaker fluorescence intensity is indicative of a more compact protein structure which exposes fewer hydrophobic sites on which ANS may bind.

All samples contained 0.05 mg/ml (3.2 μM) IL-21, 10 mM buffer (which buffer and pH is shown in the figure) and when indicated, 50 mM Na₂SO₄. 16 μM ANS was added just before measurement.

ANS fluorescence spectra were recorded on a Varian Cary Eclipse Fluorescence Spectrophotometer equipped with thermostated cell (Varian Cary Single cell Peltier accessory). Fluorescence spectrophotometer settings and performance are as follows:

Samples are excited at 390 nm. Both excitation and emission slits were set to 5 nm and photomultiplicator (PMT) and temperature were set to “high” and 20° C., respectively. Emission spectra are collected between 400-700 nm. 3 spectra are collected and averaged for each sample.

The emission maximum wavelength (λₘₐₓ) was evaluated by first derivative calculation of the emission spectrum after subtracting appropriate blank.

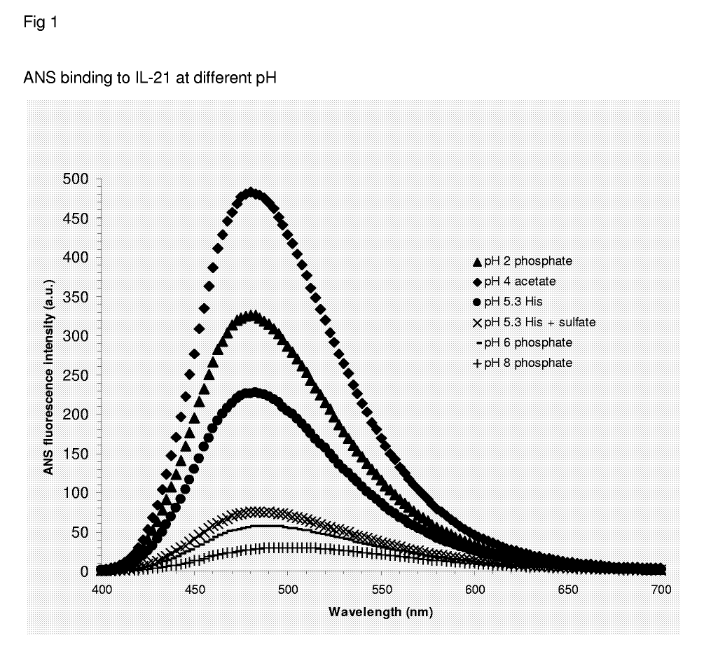

The data in FIG. 1 show ANS spectra for IL-21 at different pH and in different buffers. The data clearly show that IL-21 obtains a more compact structure as pH is increased. Notably, the addition of sulphate gives rise to a significant increase in the compactness of the IL-21 structure. It is therefore concluded that sulphate stabilises the structure of IL-21.

### Example 2

### Far-UV Circular Dichroism (CD)

Far-UV CD spectra of proteins reflect the asymmetry in the peptide bonds, which in turn reflects the secondary structure of the protein. Far-UV spectra of proteins with predominantly α-helix conformation are characterised by two distinct minima at 208 and 222 nm, by a positive peak in the 190-195 nm region, and by a cross-over from the negative to the positive region above 172 nm. The effect of sulphate on the secondary structure of IL-21 was investigated at different pH.

The spectra were recorded on a Jasco PTC-4238 spectropolarimeter fitted with a temperature controller. The scans shown represent an average of three spectra obtained by collecting data at 0.1 nm intervals with a response time of 4 s, and with a scanning rate of 20 nm/min. The spectra were corrected for blank, and the spectra were obtained from 180 to 250 nm. Protein concentration were 10 mg/ml

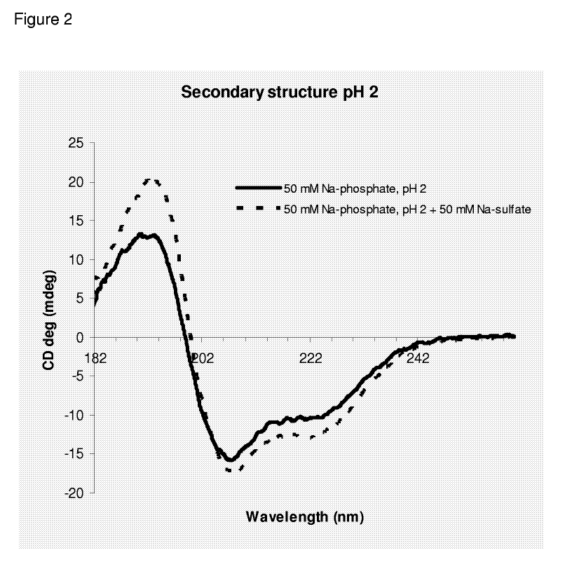

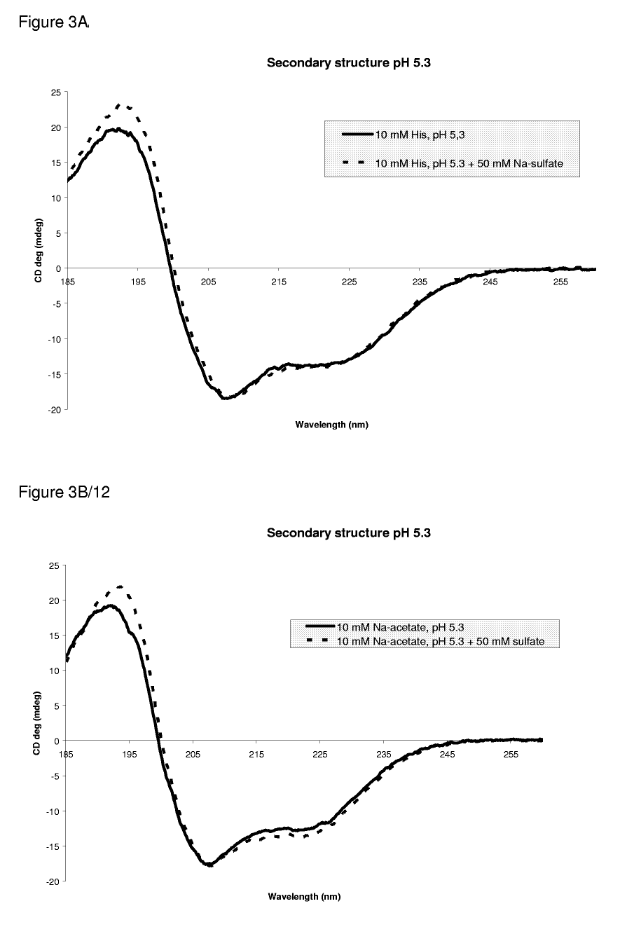

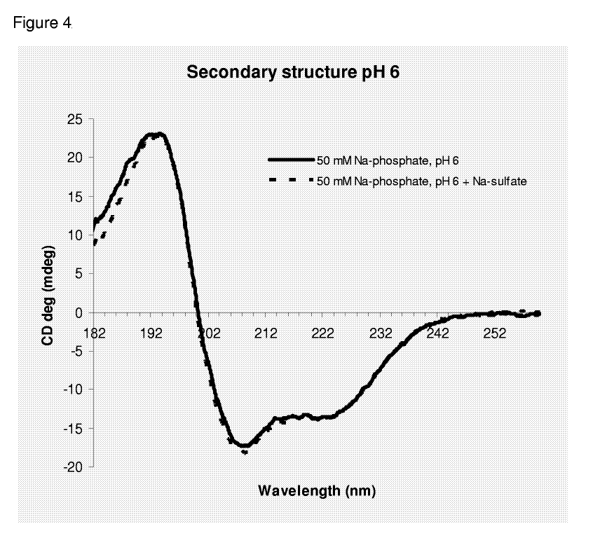

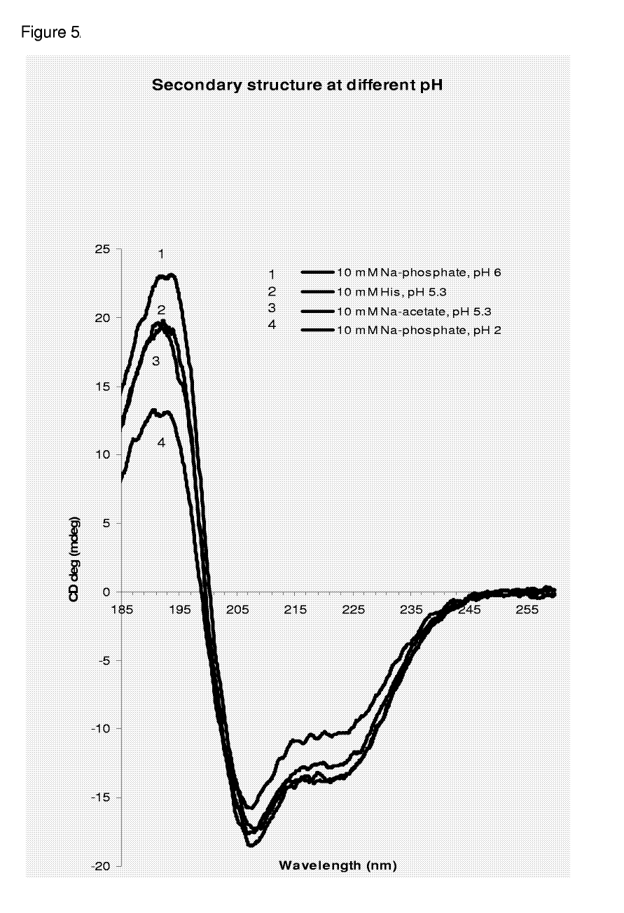

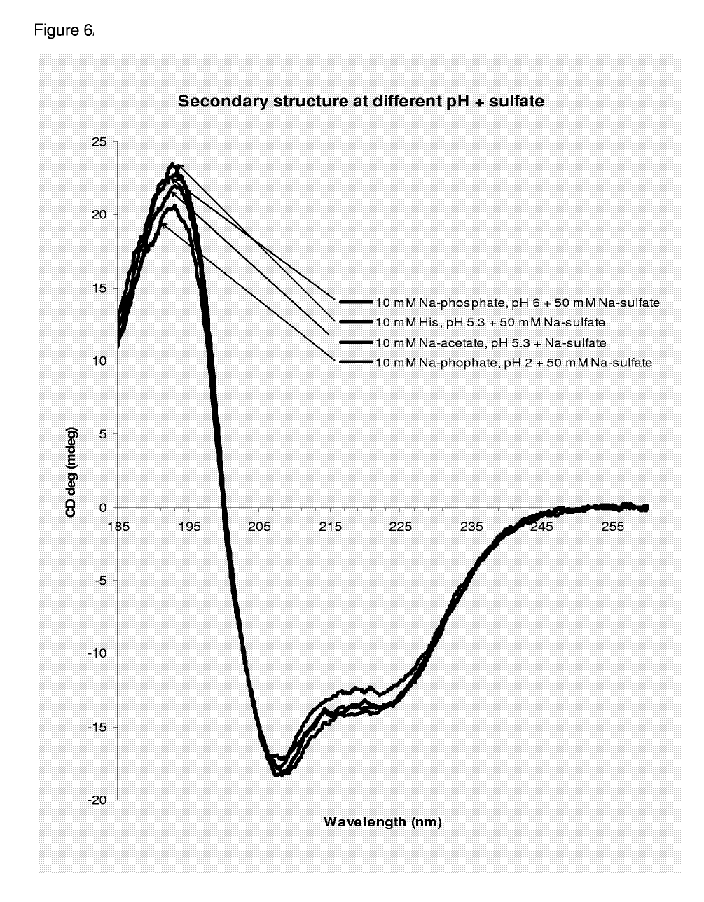

FIG. 2 shows the spectra at pH 2 in phosphate buffer with and without sulphate. At pH 2, without sulphate, IL-21 exists in a equilibrium between α-helix and random coil. However, the addition of sulphate clearly favours the α-helix confirmation. FIGS. 3 and 4 show that in particular at pH 5.3 but also at pH 6.0, addition of sulphate increases the 109-195 nm peaks which indicates an increased amount of α-helix structure. The data from FIGS. 2-4 are also presented in FIGS. 5 and 6, where FIG. 5 shows the far-UV CD spectra of IL-21 at pH 2-6 without sulphate while FIG. 6 shows the far-UV CD spectra of IL-21 in the same pH interval with sulphate added. In a comparison of FIGS. 5 and 6 it is striking that the addition of sulphate makes the secondary structure of IL-21 more uniform over a broad pH range.

It is therefore concluded that addition of sulphate to IL-21 compositions increases the α-helix content of the structure of IL-21 over a broad pH range.

### Example 3

### Near-UV Circular Dichroism (CD)

Near-UV CD reflects the tertiary structure of a protein. The higher the amplitude of the near-UV spectra, the higher the amount of tertiary structure of the protein. The near-UV spectra of IL-21 were obtained at pH 2 to 6 in various buffers with and without the addition of sulphate.

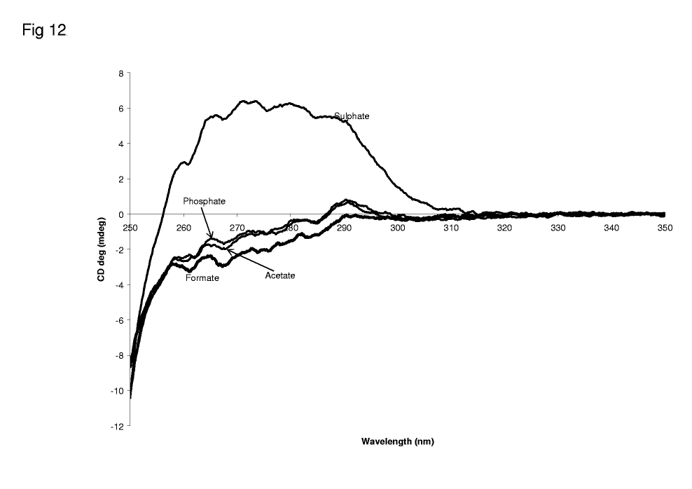

The spectra were recorded on a Jasco PTC-4238 spectropolarimeter fitted with a temperature controller. The scans shown represent an average of three spectra obtained by collecting data at 0.1 nm intervals with a response time of 4 s, and with a scanning rate of 20 nm/min. The spectra were corrected for blank, and the spectra were obtained from 250 to 350 nm. Protein concentration were 10 mg/ml, except for the experiments shown in FIG. 12, wherein the protein concentration was 2 mg/ml.

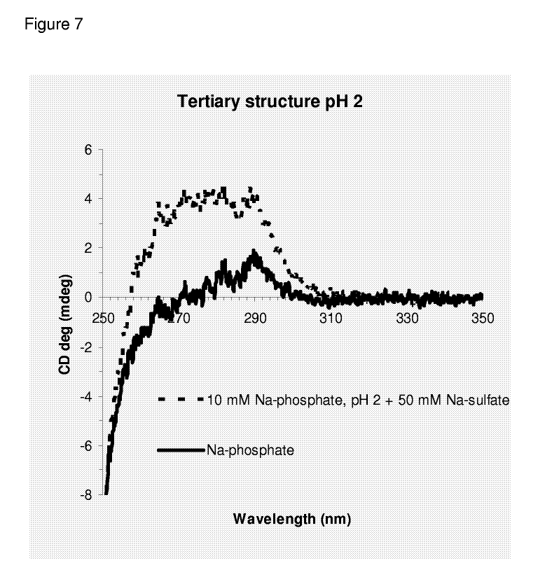

FIG. 7 shows near-UV CD spectra for IL-21 at 10 mg/ml at pH 2 (phosphate buffer) with and without the addition of 50 mM sulphate. The figure clearly shows that the addition of sulphate increases the amount of tertiary structure of IL-21.

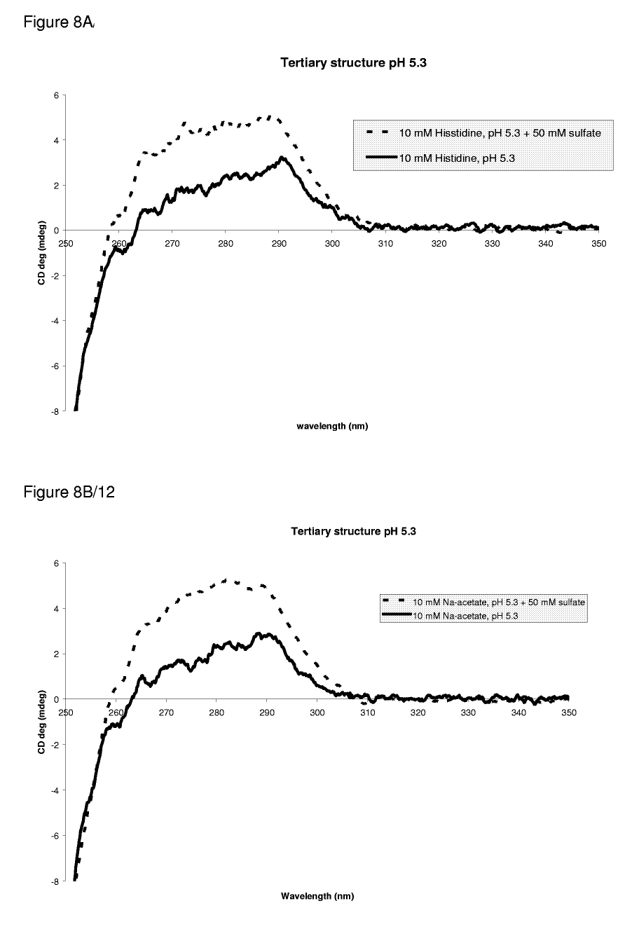

FIG. 8A shows near-UV CD spectra for IL-21 at 10 mg/ml at pH 5.3 (histidine buffer) with and without the addition of 50 mM sulphate. The figure clearly shows that the addition of sulphate increases the amount of tertiary structure of IL-21. FIG. 8B shows near-UV spectra for IL-21 at 10 mg/ml at pH 5.3 (acetate buffer) with and without the addition of 50 mM sulphate. The figure clearly shows that the addition of sulphate increases the amount of tertiary structure of IL-21. It is evident form the data depicted in FIGS. 8A and 8B that independent of the buffer, the addition of sulphate results in an increase in the amount of tertiary structure of IL-21.

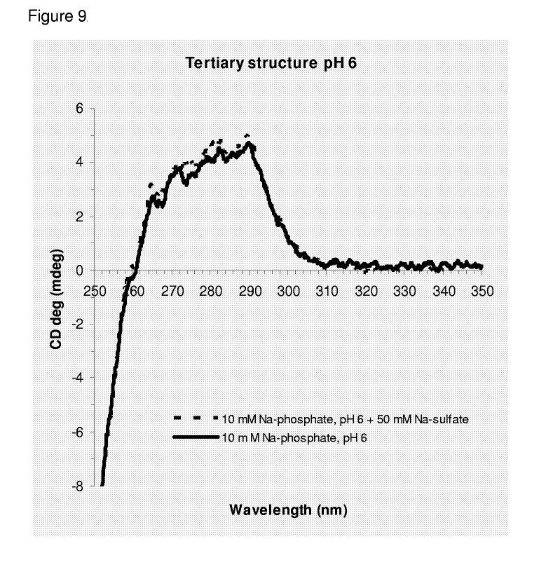

FIG. 9 shows near-UV CD spectra for IL-21 at 10 mg/ml at pH 6.0 (phosphate buffer) with and without the addition of 50 mM sulphate. The figure shows that there is a small, but significant increase in the amount of tertiary structure of IL-21 at this pH.

FIG. 12 shows near-UV CD spectra for IL-21 at 2 mg/ml at pH in various buffers. The CD spectra for the sulphate containing buffer is dramatically different from the other spectra with a change from a weak negative to a much more positive CD.

### Example 4

### Differential Scanning Calorimetry (DSC)

Differential scanning calorimetry measures the unfolding or melting temperature of a protein. A well-defined, narrow peak is indicative of a compact and defined structure and structural compactness of a protein, whereas a broad peak is indicative of a more loose structure.

Capillary DSC was conducted with a Microcal VP-DSC calorimeter. The samples were scanned from 10 to 110° C., using a scan rate at 3° C./min. The sample was run with its individual buffer as reference. Buffer-Buffer reference curves were subtracted from the sample scans to get the final sample specific data. The samples contained 10 mg/ml IL-21, mM histidine at pH 5.3, and when indicated, 50 mM Na₂SO₄.

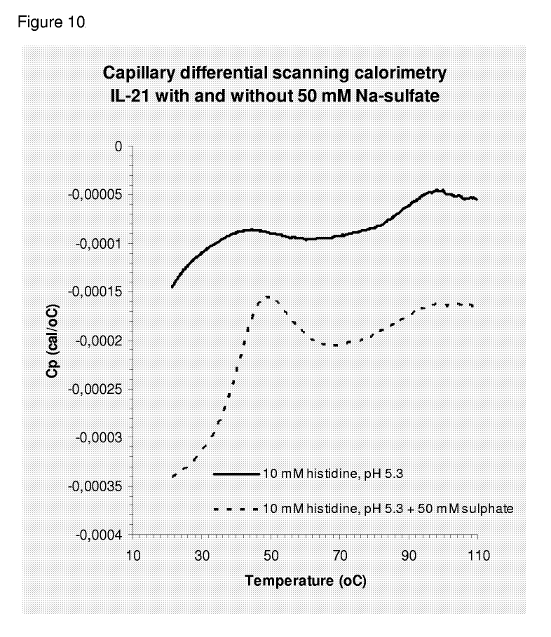

The data shown in FIG. 10 shows that the addition of sulphate to IL-21 results in the appearance of a peak in the DSCscan. This shows that sulphate increases the structural compactness of IL-21.

### Example 5

### Preparation of a Soluble Formulation of IL-21 10 mg/ml at pH 5.3 with Histidine 10 mM

A stock solution of IL-21 2.4% (w/v) was combined with a stock solution of histidine 1.52% (w/v). pH of the solution was adjusted to 5.3 with HCl and NaOH and water was added to dilute the formulation to the final volume. The formulation was sterilized by filtration using a 0.22 μm filter and aseptically filled into sterilized vials.

### Example 6

### Preparation of Four Soluble Formulations of IL-21 10 mg/ml at pH 5.3 with Histidine 10 mM and Varying Amounts of Sodium Sulphate (3, 10, 50 and 150 mM)

A stock solution of IL-21 2.4% (w/v) was combined with a stock solution of histidine 1.52% (w/v). A stock solution of sodium sulphate 10.6% (w/v) was added. pH of the solution was adjusted to 5.3 with HCl and NaOH and water was added to dilute the formulation to the final volume. The formulation was sterilized by filtration using a 0.22 μm filter and aseptically filled into sterilized vials.

The procedure was repeated four times while adding different amounts of the sodium sulphate stock solution.

### Example 7

### Preparation of a Soluble Formulation of IL-21 10 mg/ml at pH 5.3 with Mannitol 4.7% (w/v) and Histidine 10 mM

A stock solution of IL-21 2.4% (w/v) was combined with a stock solution of mannitol 23.6% (w/v) and a stock solution of histidine 1.52% (w/v). pH of the solution was adjusted to 5.3 with HCl and NaOH and water was added to dilute the formulation to the final volume. The formulation was sterilized by filtration using a 0.22 μm filter and aseptically filled into sterilized vials.

### Example 8

### Preparation of Soluble Formulations of IL-21 10 mg/ml at pH 5.3 with Mannitol 4.7% (w/v), Histidine 10 mM and Varying Amounts of Sodium Sulphate (3, 10, 50 and 150 mM)

A stock solution of IL-21 2.4% (w/v) was combined with a stock solution of mannitol 23.6% (w/v) and a stock solution of histidine 1.52% (w/v). A stock solution of sodium sulphate 10.6% (w/v) was added. pH of the solution was adjusted to 5.3 with HCl and NaOH and water was added to dilute the formulation to the final volume. The formulation was sterilized by filtration using a 0.22 μm filter and aseptically filled into sterilized vials.

The procedure was repeated four times while adding different amounts of the sodium sulphate stock solution.

### Example 9

### Preparation of Soluble Formulations of IL-21 10 mg/ml at pH 5.3 with Histidine 10 mM, Sodium Sulphate 50 mM and Various Amounts of Sodium Chloride (50 and 150 mM)

A stock solution of IL-21 2.4% (w/v) was combined with a stock solution of histidine 1.52% (w/v) and a stock solution of sodium chloride 4.38% (w/v). A stock solution of sodium sulphate 10.6% (w/v) was added. pH of the solution was adjusted to 5.3 with HCl and NaOH and water was added to dilute the formulation to the final volume. The formulation was sterilized by filtration using a 0.22 μm filter and aseptically filled into sterilized vials.

The procedure was repeated two times while adding different amounts of the sodium chloride stock solution.

### Example 10

### Preparation of Soluble Formulations of IL-21 10 mg/ml at pH 5.3 with Mannitol 4.7% (w/v), Histidine 10 mM, Sodium Sulphate 50 mM and Various Amounts of Sodium Chloride (50 and 150 mM)

A stock solution of IL-21 2.4% (w/v) was combined with a stock solution of mannitol 23.6% (w/v), a stock solution of histidine 1.52% (w/v) and a stock solution of sodium chloride 4.38% (w/v). A stock solution of sodium sulphate 10.6% (w/v) was added. pH of the solution was adjusted to 5.3 with HCl and NaOH and water was added to dilute the formulation to the final volume. The formulation was sterilized by filtration using a 0.22 μm filter and aseptically filled into sterilized vials.

The procedure was repeated two times while adding different amounts of the sodium chloride stock solution.

### Example 11

### Storage Stability

The vials containing formulations from example 5 to 11 were placed at different temperatures: 37° C., 25° C., 15° C., 5° C. and −20° C. Samples for analysis were drawn at various time points. The content of “aggregates” (including covalent dimers and other high molecular weight impurities) in the IL-21 formulations was determined by Size-Exclusion HPLC on a TSK column. The percentage of aggregates is based on the total area of peaks eluting prior to the IL-21 main peak, relative to the total area within the included volume.

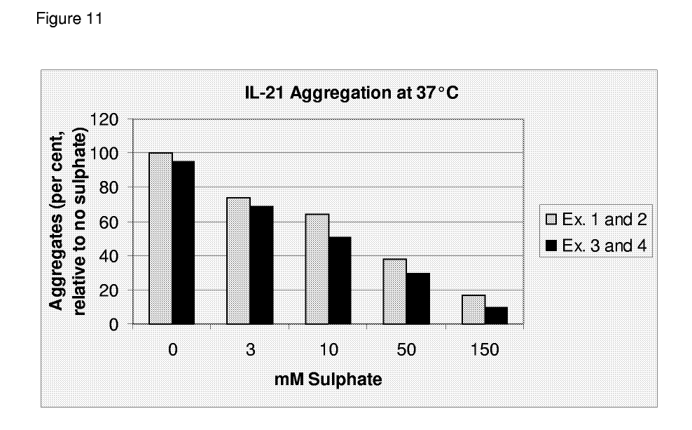

Results from samples stored for one month at 37° C., normalized using the formulation in Example 5 (i.e. the IL-21 formulation without sulphate or mannitol) as reference, are listed in Table 1. The data are also depicted in FIG. 11.

TABLE 1
Normalized aggregate formation after one month at 37° C. in
formulations of IL-21 described in Examples 5-10. The reference
from Example 5 is set to 100

| | | | Ex. 9 | Ex. 10 | | Ex. 10 |
|---|---|---|---|---|---|---|
| | Ex. 5 | Ex. 7 | Histi- | Histidine | Ex. 9 | Histidine |
| Sodium | and 6 | and 8 | dine | Mannitol | Histidine | Mannitol |
| sulphate | Histi- | Histidine | NaCl | NaCl | NaCl | NaCl |
| (mM) | dine | Mannitol | 50 mM | 50 mM | 150 mM | 150 mM |
| 0 | 100.0 | 95.0 | | | | |
| 3 | 73.7 | 68.9 | | | | |
| 10 | 64.1 | 50.4 | | | | |
| 50 | 37.7 | 29.7 | 37.2 | 27.9 | 34.3 | 26.8 |
| 150 | 17.0 | 9.8 | | | | |

The data from Table 1 (or depicted in FIG. 11) show that sulphate has a positive effect on the storage stability of IL-21. The data also show this positive effect to be dependent on the concentration of sulphate. Moreover, the data show that chloride has a positive effect on the storage stability, too, and that this effect is also dependent on the chloride concentration. Finally, the data show that an additional stabilising effect is achieved by adding both sulphate and chloride to an IL-21 composition. It is thus established that the effect of sulphate on the structure of IL-21 demonstrated by the biophysical experiments disclosed in examples 1-4 is reflected in an increased stability of IL-21 compositions.

1. A composition comprising an IL-21 and sulphate, provided any ammonium ions, if present, are not present in a molar amount which is twice the molar amount of sulphate.

2. The composition of claim 1, wherein sulphate is present in a concentration above 1 mM.

3. The composition according to claim 2, wherein the pH is between around 1 and around 10.

4. The composition according to claim 1, wherein the sequence of the IL-21 comprises amino acid nos. 32-162 of SEQ ID NO: 1.

5. The composition according to claim 1, wherein the sequence of the IL-21 comprises amino acid nos. 30-162 of SEQ ID NO: 1.

6. The composition according to claim 5, wherein the sequence of the IL-21 comprises SEQ ID NO: 1.

7. The composition according to claim 1, wherein the sequence of the IL-21 comprises SEQ ID NO: 2.

8. The composition according to claim 2, wherein the sequence of the IL-21 is represented by SEQ ID NO: 1.

9. The composition according to claim 2, wherein the sequence of the IL-21 is represented by amino acid nos. 32-162 of SEQ ID NO: 1.

10. The composition according to claim 2, wherein the sequence of the IL-21 is represented by amino acid nos. 30-162 of SEQ ID NO: 1.

11. The composition according to claim 2, wherein the sequence of the IL-21 is represented by SEQ ID NO: 2.

12. The composition according to claim 2, wherein said composition is a pharmaceutical composition.

13. The pharmaceutical composition according to claim 12, wherein the composition is isotonic.

14. The composition according to claim 9, wherein the composition is an isotonic pharmaceutical composition.

15. The composition according to claim 1, which comprises chloride.

16. The composition according to claim 15, wherein said composition does not comprise ammonium.

17. The composition according to claim 16, wherein said composition does not comprise copper.

18. A pharmaceutical composition comprising IL-21 and sulphate ions.

19. The pharmaceutical composition according to claim 18, wherein the composition is isotonic.

20. (canceled)

21. The pharmaceutical composition according to claim 19, wherein any ammonium ions, if present, are not present in a molar amount which is twice the molar amount of sulphate.

22. The pharmaceutical composition according to claim 21, which comprises chloride.

23. The pharmaceutical composition according to claim 22, wherein said composition does not comprise ammonium.

24. The pharmaceutical composition according to claim 23, wherein said composition does not comprise copper.

25. A composition comprising about 10 mg/ml of an IL-21 and 10 mM Na₂SO₄ which is buffered with histidine at pH 5.3.

26. The composition according to claim 25, which composition is a pharmaceutical composition.

27. The composition according to claim 26, wherein the sequence of the IL-21 comprises amino acid nos. 32-162 of SEQ ID NO: 1.

28. The composition according to claim 26, wherein the sequence of the IL-21 comprises amino acid nos. 30-162 of SEQ ID NO: 1.

29. The composition according to claim 28, wherein the sequence of the IL-21 comprises SEQ ID NO: 1.

30. The composition according to claim 26, wherein the sequence of the IL-21 comprises SEQ ID NO: 2.

31. The composition according to claim 25, wherein the sequence of the IL-21 is represented by SEQ ID NO: 1.

32. The composition according to claim 25, wherein the sequence of the IL-21 is represented by amino acid nos. 32-162 of SEQ ID NO: 1.

33. The composition according to claim 25, wherein the sequence of the IL-21 is represented by amino acid nos. 30-162 of SEQ ID NO: 1.

34. The composition according to claim 25, wherein the sequence of the IL-21 is represented by SEQ ID NO: 2.

35. A method of stabilizing a composition comprising IL-21, the method comprising adding sulphate to said composition in an amount sufficient to detectably stabilize the IL-21 as compared to a composition lacking the sulphate.

36. A method of refolding unfolded or partially folded IL-21 in solution, the method comprising adding sulphate to said solution in an amount sufficient to stabilize the IL-21 as compared to a composition lacking the sulphate.

37. A method of purifying IL-21 from a stabilized IL-21 composition, the method comprising bringing a solution of IL-21 and a stabilizing amount of sulphate into contact with chromatographic material so as to purify the IL-21 in the composition.

38. The method according to claim 35, wherein the sequence of the IL-21 comprises amino acid nos. 32-162 of SEQ ID NO: 1.

39. The method according to claim 35, wherein the sequence of the IL-21 comprises amino acid nos. 30-162 of SEQ ID NO: 1.

40. The method according to claim 39, wherein the sequence of the IL-21 comprises SEQ ID NO: 1.

41. The method according to claim 35, wherein the sequence of the IL-21 comprises SEQ ID NO: 2.

42-45. (canceled)

46. A method of treating cancer, the method comprising administering to a patient in need thereof a therapeutically effective amount of a composition comprising an IL-21 and sulphate, wherein the molar amount of ammonium ions in the composition, if present, is less than twice the molar amount of sulphate.

47. The method according to claim 46, wherein said cancer is selected amongst renal cell carcinoma, colorectal cancer, melanoma and non-Hodgkins lymphoma.

48-57. (canceled)


SEQUENCE LISTING (2 sequences)

SEQ ID NO: 1 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 2 - protein, 134 aa, Artificial
  IL-21 variant
MQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS